(19) United States
(12) Patent Application Publication
(10) Pub. No.: US 2013/0012891 A1
(43) Pub. Date: Jan. 10, 2013

(54) SKIN-TREATMENT AND DRUG-DELIVERY DEVICES

(76) Inventors: Yossi Gross, Moshav Mazor (IL); Noah Amit, Haifa (IL)

(21) Appl. No.: 13/176,072

(22) Filed: Jul. 5, 2011

(51) Int. Cl. A61M 35/00

(52) U.S. Cl. 604/289

(57) ABSTRACT

Apparatus for use with skin of a subject is provided, including a mount, a first zone of which is adhesively adherable to the skin. A second zone is not adhesively adherable to the skin. The mount is disposable in one shape and in another shape, at least one of the shapes being an unconstrained shape, the mount being deformable from the one shape to the other shape in response to application of a deforming force to the mount. When the mount moves toward the unconstrained shape, the movement is sudden regardless of a speed of application of the deforming force prior to the sudden movement. A treatment unit is provided, coupled to the mount, positioned with respect to the mount such that the treatment unit is moved into a position to apply a treatment to the subject by the mount moving toward the unconstrained shape. Other applications are also described.

### FIELD OF THE INVENTION

The present invention relates to skin-treatment and drug-delivery devices and methods, specifically devices and methods for the treatment of skin wrinkles and for administering drugs. In particular, the invention relates to applying lifting and pressing forces to the skin.

### BACKGROUND

A significant feature of aging is the loss of skin elasticity and the formation of skin wrinkles. Wrinkles are considered undesirable by some individuals, and significant demand exists for wrinkle-reducing treatments.

### SUMMARY OF THE INVENTION

In some applications of the invention, a resilient structure is adhered to the skin of a subject in a manner such that a wrinkle is lifted in a superficial direction and held for a period of hours. Repetition of this procedure, typically on a daily basis, reduces the depth and, therefore, the appearance of the wrinkle.

In another application of the invention, a resilient structure is adhered to the skin of a subject in a manner such that a drug delivery system is held tightly against the surface of the skin, reducing leakage commonly associated with drug delivery.

There is therefore provided, in accordance with an application of the present invention, apparatus for use with skin of a subject, including:

a mount, a first zone of which is adhesively adherable to the skin and a second zone of which is not adhesively adherable to the skin, the mount being disposable in one shape and in another shape, at least one of the shapes being an unconstrained shape, the mount being deformable from the one shape to the other shape in response to application of a deforming force to the mount, and configured such that when the mount moves toward the unconstrained shape, the movement is sudden regardless of a speed of application of the deforming force prior to the sudden movement; and

a treatment unit coupled to the mount, positioned with respect to the mount such that the treatment unit is moved into a position to apply a treatment to the subject by the mount moving toward the unconstrained shape.

In an application, the skin of the subject to which the first zone of the mount is adhesively adherable has a plane, and the treatment unit is positioned with respect to the mount such that the mount moving toward the unconstrained shape, moves the treatment unit in a direction that is substantially orthogonal to the plane.

In an application, the one shape is the unconstrained shape, and the mount has sufficient resilience to return toward the one shape after removal of the deforming force.

In an application, the first zone of the mount is configured to be disposed more superficially when the mount is adhered to the skin and in the unconstrained shape than when the mount is adhered to the skin and in the other shape.

In an application, the other shape is the unconstrained shape and the one shape is an unconstrained shape, and the mount is configured such that it remains in the other shape after removal of the deforming force.

In an application, the first zone of the mount is configured to be disposed more superficially when the mount is adhered to the skin and is in the one shape than when the mount is adhered to the skin and is in the other shape.

In an application, the first zone of the mount is configured to be disposed more superficially when the mount is adhered to the skin and is in the other shape than when the mount is adhered to the skin and is in the one shape.

In an application:

the treatment unit includes an adhesive, and

the adhesive is configured to apply the treatment by being positioned with respect to the mount such that when the mount is adhered to the skin, the mount moving toward the unconstrained shape moves the adhesive superficially.

In an application, the apparatus further includes a power supply and at least one electrode, and the power supply is configured to drive the electrode to apply an electrical current to the skin of the subject.

In an application, the apparatus is configured for use with a drug, and the power supply is configured to drive the electrode to apply an iontophoretic current which drives the drug into the skin of the subject.

In an application, the power supply is configured to drive the electrode to electrically stimulate the skin of the subject.

In an application, the treatment unit includes a light source, the light source being configured to illuminate the skin of the subject with red light.

In an application:

the treatment unit includes a power supply and at least one electrode,

the treatment unit is positioned with respect to the mount such that the mount moving toward the unconstrained shape is configured to push the electrode against the skin of the subject, and

the electrode is configured to apply an electrical current to the skin of the subject.

In an application, the power supply is configured to drive the electrode to apply an iontophoretic current which drives the drug into the skin of the subject.

In an application, the power supply is configured to drive the electrode to electrically stimulate the skin of the subject.

In an application, the treatment unit includes a light source, the light source being configured to illuminate the skin of the subject with red light.

In an application:

the treatment unit includes at least one needle, and

the treatment unit is configured such that the treatment unit being moved into the position to apply the treatment is configured to push the needle toward the skin of the subject.

In an application, the at least one needle includes at least one hollow needle, and the treatment unit is configured to administer a drug via the at least one hollow needle.

In an application, the apparatus includes at least one seal shaped to define a lumen, each seal being disposed around a base of a respective hollow needle.

In an application, the first zone of the mount includes at least part of a skin-facing side of the mount, configured to face skin of the subject, and the second zone of the mount includes an outward-facing side of the mount, configured to face away from skin of the subject.

In an application, the first zone of the mount is disposed in a central region of the skin-facing side of the mount.

In an application, the first zone of the mount is not disposed at an edge of the skin-facing side of the mount.

In an application, the first zone of the mount is disposed in a peripheral region of the skin-facing side of the mount.

In an application, the first zone of the mount is not disposed in a central region of the skin-facing side of the mount.

In an application, the mount is configured such that it is deformable from the one shape to the other shape only when the deforming force is at least 20 mN.

In an application, the mount is configured such that it is deformable from the one shape to the other shape only when the deforming force is at least 100 mN.

In an application, the mount is configured such that when at least one element of the mount is stationary and the mount moves to the unconstrained shape, the treatment unit moves at least 3 mm.

In an application, the mount is configured such that when the element of the mount is stationary and the mount moves to the unconstrained shape, the treatment unit moves at least 5 mm.

In an application, the mount is configured such that when the element of the mount is stationary and the mount moves to the unconstrained shape, no part of the mount moves greater than 40 mm.

In an application, the mount is configured such that when the element of the mount is stationary and the mount moves to the unconstrained shape, no part of the mount moves greater than 25 mm.

In an application, the mount is configured such that it is deformable from the one shape to the other shape only when the deforming force is at least 20 mN, and the mount is configured such that it is deformable from the one shape to the other shape even when the deforming force is less than 2000 mN.

In an application, the apparatus includes at least one structural ridge, the at least one structural ridge configured to increase a resilience of the mount.

In an application, the structural ridge is configured to contribute to the suddenness of the movement.

In an application, the mount includes the structural ridge, and wherein the mount and the structural ridge comprise a continuous piece of a material.

In an application, the at least one structural ridge is coupled to the mount.

In an application, the apparatus includes a drug reservoir, coupled to the treatment unit, and configured to facilitate delivery of a drug to the subject.

In an application, the apparatus includes a topical drug disposed on a skin-facing side of the mount.

In an application, the apparatus includes an ultrasound transducer, configured to apply ultrasound to the skin of the subject when the mount is adhered to the skin of the subject.

In an application, the mount includes a plurality of mounts and the adhesive includes a respective plurality of adhesives, and the apparatus further includes a kit containing the plurality of mounts and adhesives.

There is further provided, in accordance with an application of the present invention, apparatus for use with skin of a subject, including:

a reservoir;

a plurality of seals, each seal shaped to define a lumen, one end of each lumen being open; and

a respective plurality of hollow needles, each needle shaped to form a conduit, a part of each hollow needle being disposed within the lumen of a respective seal, each conduit being in fluid communication with the reservoir.

In an application, the apparatus includes a baseplate to which the plurality of seals are coupled.

In an application, the baseplate includes at least part of a wall of the reservoir.

There is further provided, in accordance with an application of the present invention, a method for use with a wrinkle of skin of a subject, the method including:

adhering apparatus to the wrinkle of the subject, the apparatus including a mount and an adhesive;

deforming the mount from one shape toward another shape by applying a deforming force to the mount, at least one of the shapes being an unconstrained shape; and

inducing a sudden movement of the mount toward the unconstrained shape, such that the adhesive moves superficially with respect to the skin, the suddenness of the movement being regardless of a speed of application of the deforming force prior to the sudden movement.

In an application, deforming the mount from the one shape toward the other shape includes deforming the mount from an unconstrained shape toward a constrained shape, and wherein inducing sudden movement of the mount toward the unconstrained shape includes removing the deforming force, the mount having sufficient resilience to return toward the unconstrained shape after removal of the deforming force.

In an application, deforming the mount from the one shape toward the other shape includes deforming the mount from one unconstrained shape toward another unconstrained shape.

In an application, the method further includes delivering a drug to the subject using the apparatus.

In an application, delivering the drug to the subject includes applying the drug topically, the drug being disposed on a skin-facing side of the apparatus.

In an application, delivering the drug to the subject includes delivering the drug by iontophoresis.

In an application, the method further includes applying ultrasound to the skin of the subject while the apparatus is adhered to the wrinkle of the subject.

In an application, the method further includes applying electrical current to the skin of the subject while the apparatus is adhered to the wrinkle of the subject.

In an application, the method further includes applying red light to the skin of the subject while the apparatus is adhered to the wrinkle of the subject.

There is further provided, in accordance with an application of the present invention, a method for use with skin of a subject, the method including:

adhering apparatus to the skin of the subject, the apparatus including a mount and a treatment unit coupled to the mount; and

deforming the mount from one shape to another shape by applying a deforming force to the mount, at least one of the shapes being an unconstrained shape; and

inducing a sudden movement of the mount toward the unconstrained shape, such that the treatment unit is moved into a position to apply a treatment to the subject.

In an application, the skin of the subject to which the apparatus is adhered has a plane, and inducing the sudden movement of the mount toward the unconstrained shape such that the treatment unit is moved into a position to apply a treatment to the subject, includes moving the treatment unit in a direction that is substantially orthogonal to the plane.

In an application, deforming the mount from the one shape into the other shape includes deforming the mount from an unconstrained shape toward a constrained shape, and inducing sudden movement of the mount toward the unconstrained shape includes removing the deforming force, the mount having sufficient resilience to return toward the unconstrained shape after removal of the deforming force.

In an application, inducing the sudden movement of the mount such that the treatment unit is moved into the position to apply the treatment to the subject includes inducing a sudden superficial movement of the treatment unit, the treatment unit including an adhesive.

In an application, deforming the mount from the one shape into the other shape includes deforming the mount from one unconstrained shape into another unconstrained shape, the mount being configured such that it remains in the other unconstrained shape after removal of the deforming force.

In an application, inducing the sudden movement of the mount such that the treatment unit is moved into the position to apply the treatment to the subject includes inducing a sudden movement of the mount such that the treatment unit is moved superficially, the treatment unit including an adhesive.

In an application, inducing the sudden movement of the mount such that the treatment unit is moved into the position to apply the treatment to the subject includes causing the treatment unit to be pressed against the skin of the subject by inducing the sudden movement of the mount.

In an application, the treatment unit includes a plurality of needles, and causing the treatment unit to be pressed against the skin of the subject includes causing the needles to be pressed into the skin of the subject.

In an application, the method further includes administering a drug to the subject via the needles.

In an application, the treatment unit includes at least one electrode, and inducing the sudden movement of the mount such that the treatment unit is pressed against the skin of the subject includes causing the electrode to be pressed against the skin of the subject by inducing the sudden movement of the mount.

In an application, the method further includes administering a drug to the subject by iontophoresis, using the electrode.

There is further provided, in accordance with an application of the present invention, apparatus for use with skin of a subject, including:

a mount, shaped to provide an opening and being adhesively adherable to the skin in a manner that maintains a reduction of a wrinkle on the skin;

a glue, configured to reduce flexibility of the skin when adhered to the skin; and

an applicator, configured to apply the glue to the skin via the opening provided by the mount.

In an application, the mount is disposable in one shape and in another shape, at least one of the shapes being an unconstrained shape, the mount is configured to reduce the wrinkle by being deformable from the one shape to the other shape in response to application of a deforming force to the mount.

In an application, the mount is configured such that when the mount moves toward the unconstrained shape, the movement is sudden regardless of a speed of application of the deforming force prior to the sudden movement

In an application, the opening provided by the mount has one profile and another profile, the mount in the one shape defining the opening having the one profile and the mount in the other shape defining the opening having the other profile.

In an application, the mount is configured such that when the mount is adhered to the skin and is in the one shape, a zone of the mount defining the opening is disposed more superficially than when the mount is adhered to the skin and is in the other shape.

In an application, the glue includes one or more substances selected from the group consisting of: cyanoacrylate, latex, and collagen.

In an application, the applicator includes an applicator selected from the group consisting of: a syringe, a brush, a spreader, a dropper, a sprayer, and a pen.

There is further provided, in accordance with an application of the present invention, a method for treating skin of a subject, the method including:

reducing a wrinkle on the skin by deforming the skin;

maintaining the reduction of the wrinkle with a mount configured for adhering to the skin of the subject, the mount being shaped to provide an opening; and

reducing the flexibility of the skin by applying a glue to the skin via the opening provided by the mount.

In an application, the method further includes removing the mount from the skin after the glue has become firm, such that the glue remains on the skin.

In an application, reducing the wrinkle by deforming the skin includes flattening the wrinkle by stretching the wrinkle.

In an application, reducing the wrinkle by deforming the skin includes deforming the mount from one shape to another shape while the mount is adhered to the skin.

In an application, deforming the mount from the one shape to the other shape includes deforming the opening from one profile to another profile.

In an application, the method further includes deforming the opening from one profile to another profile.

In an application, applying the glue to the skin includes applying the glue to the skin using an applicator selected from the group consisting of: a syringe, a brush, a spreader, a dropper, a sprayer, a pen.

In an application, the method further includes applying a drug to the skin of the subject while the mount is adhered to the skin.

In an application, the method further includes applying ultrasound to the skin of the subject while the mount is adhered to the skin.

In an application, the method further includes applying electrical current to the skin of the subject while the mount is adhered to the skin.

There is further provided, in accordance with an application of the present invention, apparatus for use with skin of a subject, including:

a mount, adhesively adherable to the skin of the subject, having an unconstrained shape, the mount being deformable from the unconstrained shape to a constrained shape, and biased toward assuming the unconstrained shape;

an adhesive, disposed on a first part of the mount; and

a treatment unit, coupled to a second part of the mount, and positioned with respect to the mount and configured such that when the mount is adhered to the skin of the subject, the adhesive is disposed more superficially than is a skin-contact portion of the treatment unit.

In an application, the treatment unit includes a plurality of needles.

In an application, the treatment unit includes at least one electrode.

### BRIEF DESCRIPTION OF THE DRAWINGS

FIGS. 1A-B are schematic illustrations of a wrinkle and of apparatus for treating the wrinkle, in accordance with respective applications of the invention;

FIGS. 2A-C and 3A-C are schematic illustrations of a wrinkle being treated, in accordance with respective applications of the invention;

FIGS. 4, 5A-B, 6A-B, and 7 are schematic illustrations of various configurations of the apparatus of FIGS. 1-3, in accordance with respective applications of the invention;

FIGS. 8A-D are schematic illustrations of structural ridges incorporated into the apparatus of FIGS. 1-7, in accordance with an application of the invention;

FIGS. 9A-B are schematic illustrations of circuitry for use with the apparatus of FIGS. 1-8, in accordance with an application of the invention;

FIGS. 10A-H are schematic illustrations of apparatus for facilitating passage of a substance through the skin, in accordance with another application of the invention;

FIGS. 11A-H are schematic illustrations of apparatus for facilitating passage of a substance through the skin, in accordance with another application of the invention; and

FIGS. 12A-F are schematic illustrations of a wrinkle being treated, in accordance with an application of the invention.

### DETAILED DESCRIPTION OF EMBODIMENTS

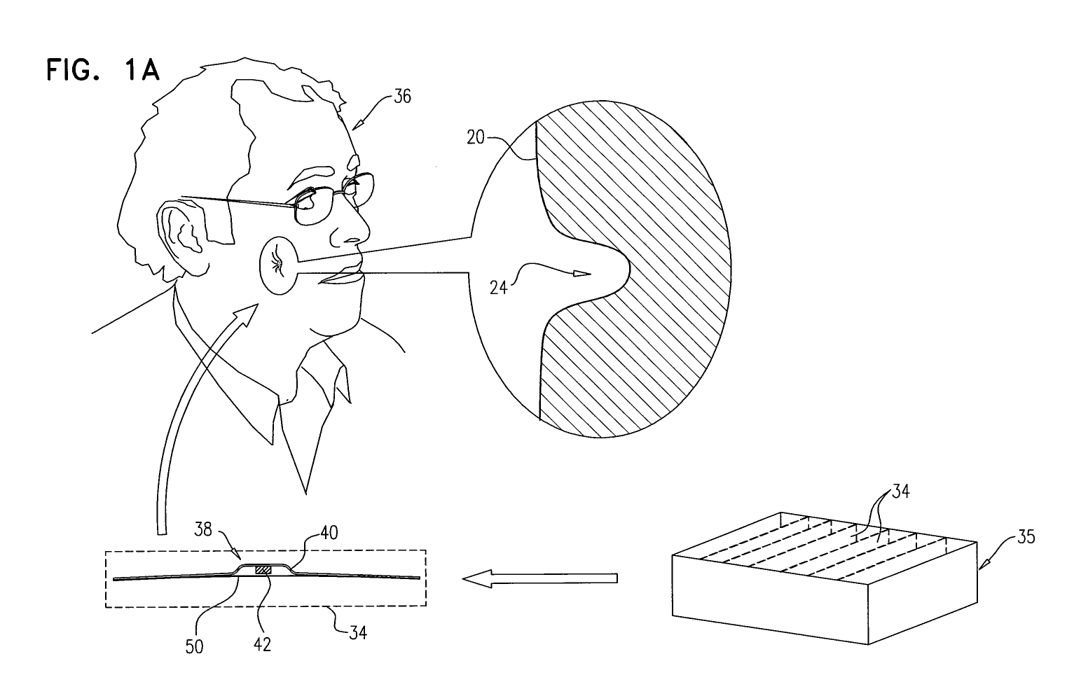

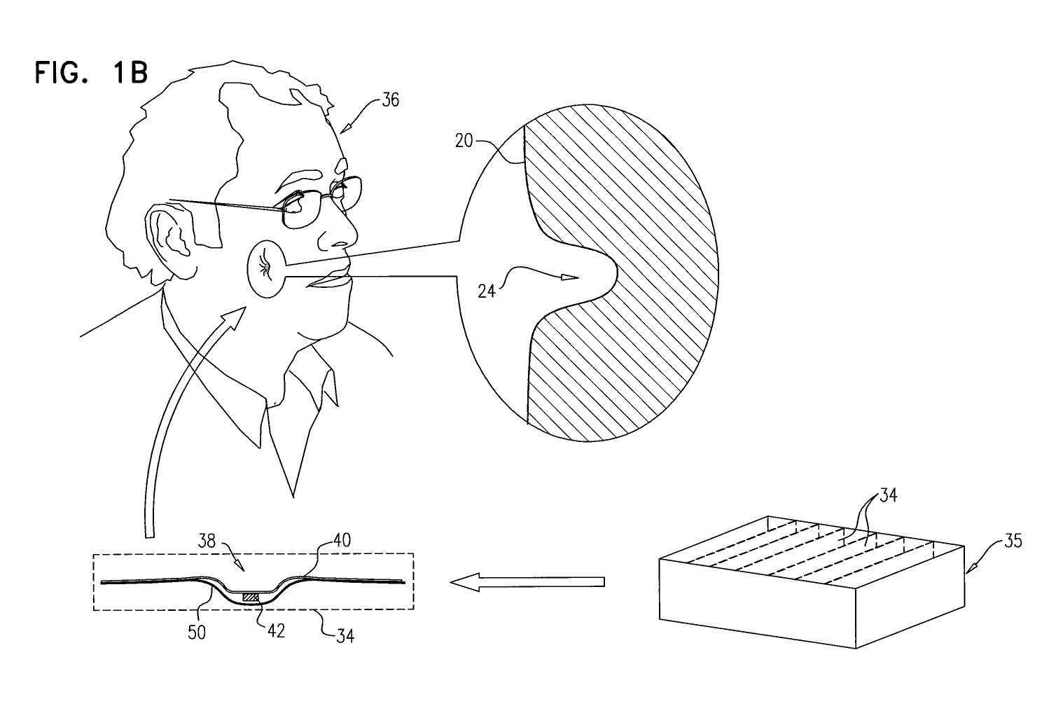

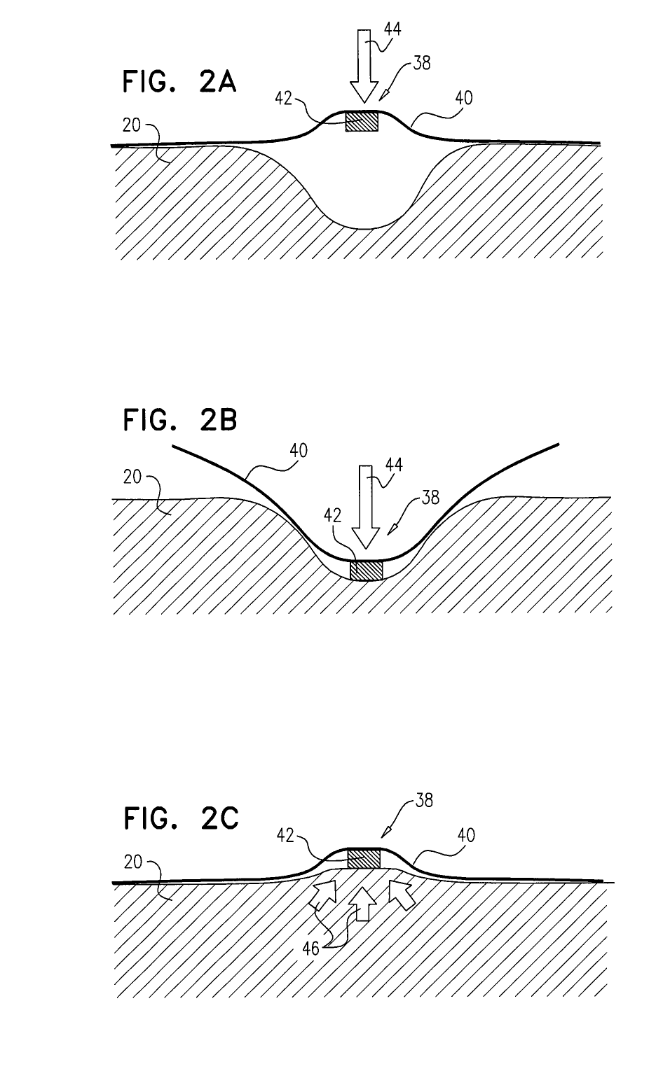

Reference is made to FIGS. 1A-B, which are schematic illustrations of apparatus 38 for application to a wrinkle 24 on skin 20 of a subject 36, in accordance with respective applications of the invention. (Use of apparatus 38 as shown in FIGS. 1A-B is described hereinbelow with reference to FIGS. 2A-3C.) Wrinkle 24 is a depressed area of skin 20 occurring, for example, around the eyes, mouth, or forehead. Apparatus 38 is supplied in a typically sealed packaging 34. For some applications, a kit 35 is provided, comprising more than one (e.g., at least 5 and/or less than 40) apparatus 38, each in a respective packaging 34.

Apparatus 38 comprises a mount 40 and a treatment unit. Mount 40 typically has two sides: a skin-facing side, which is applied to the skin, and an outward-facing side, which remains exposed to the air. For this application of the invention, the treatment unit comprises an adhesive 42, the adhesive being typically disposed in a central region of the skin-facing side of the mount and not in more peripheral regions of the skin-facing side of the mount (e.g., not at the edge of the skin-facing side of the mount). A release liner 50 is typically disposed on the skin-facing side of the mount, and is large enough to at least cover adhesive 42, but may be as large as, or larger than, mount 40. Release liner 50 protects the skin-facing side of the mount and, in particular, protects and maintains adhesive 42. Release liner 50 is removed prior to the application of apparatus 38 to the skin. Typically, the release liner comprises a material which adheres to adhesive 42 and mount 40, but does not damage these components when removed. The skin-facing side of mount 40 may comprise a therapeutic substance such as a vitamin, mineral or drug, to provide treatment of the wrinkle in addition to the lifting treatment described hereinbelow.

FIG. 1A illustrates an application of apparatus 38, prior to its application to the skin. In this application of the invention, mount 40 is concave toward adhesive 42, such that adhesive 42 is disposed within the concavity.

FIG. 1B illustrates an application of apparatus 38, prior to its application to the skin. In this application of the invention, mount 40 is convex toward adhesive 42, such that adhesive 42 is disposed opposite the concavity.

Reference is made to FIGS. 2A-C, which are side-view schematic illustrations of apparatus 38 being applied to a wrinkle 24, in accordance with one application of the invention.

Reference is now made to FIG. 2A. Apparatus 38 (as described hereinabove with reference to FIG. 1A) is applied to the skin, typically such that adhesive 42 is positioned over wrinkle 24. Mount 40 is shown in an unconstrained shape that is concave toward adhesive 42, such that adhesive 42 is disposed within the concavity. Adhesive 42 is typically disposed at or near the center of mount 40, on the skin-facing side, having properties that allow it to adhere to the skin strongly enough for effective use of this application of the invention, but not so strongly so as to make removal difficult or painful. Mount 40 comprises a resilient material that allows the mount to be flexed into a constrained shape (e.g., a bent shape, or a non-bent shape e.g., a flat shape, which is nevertheless constrained) and to return toward its unconstrained shape when released. This behavior can be described as monostable, i.e., the apparatus is stable in only one unconstrained shape.

Reference is now made to FIG. 2B. Typically, a user pushes down on apparatus 38 (i.e., applies a deforming force), deforming mount 40 and pushing adhesive 42 into wrinkle 24, as illustrated by arrow 44. The deforming force required to deform mount 40 in this manner is typically greater than 20 mN, and may be greater than 100 mN. Typically, the deforming force required to deform mount 40 is not greater than 2000 mN. Typically, the distance the adhesive moves, from when the mount is in its unconstrained shape to when the adhesive contacts the skin, is greater than 3 mm and may be greater than 5 mm. Alternatively or additionally, the user may make the wrinkle shallower prior to deformation of apparatus 38 (e.g., by pulling the skin in a direction substantially orthogonal to that of the wrinkle), thereby reducing the distance that adhesive 42 moves before contacting the skin.

Reference is now made to FIG. 2C. Once the adhesive 42 has bound to skin 20, the force indicated by arrow 44 is released, allowing mount 40 to return toward its original unconstrained shape. Due to its adhesion to adhesive 42 of apparatus 38, the skin is automatically lifted superficially, as illustrated by arrows 46. In some applications of the invention, mount 40 is configured such that movement of mount from the constrained shape to the unconstrained shape is sudden. In some applications of the invention, mount 40 is configured such that such sudden movement is accompanied by a noise, e.g., a click. The click may be useful for indicating to the user that the operation of apparatus 38 has successfully caused the desired lifting of the skin.

FIGS. 2A-C show an application in which the unconstrained shape of mount 40 is concave toward adhesive 42. Alternatively or additionally, the unconstrained shape may be any shape that disposes adhesive 42 more superficially than does the constrained shape. For example, the unconstrained shape may be flat.

Reference is made to FIGS. 3A-C, which are side-view schematic illustrations of apparatus 38 (as described hereinabove with reference to FIG. 1B) being applied to a wrinkle 24, in accordance with one application of the invention.

Reference is now made to FIG. 3A. Apparatus 38 is applied to the skin, typically such that adhesive 42 makes contact with wrinkle 24. Adhesive 42 is typically positioned within wrinkle 24. Mount 40 is shown in an unconstrained shape that is convex toward adhesive 42. Adhesive 42 is typically disposed in or near the center of mount 40, on the skin-facing side, having properties that allow it to adhere to the skin strongly enough for effective use of this application of the invention, but not so strongly so as to make removal difficult or painful. Mount 40 comprises a resilient material that typically allows the mount to be flexed between two unconstrained shapes. This behavior can be described as bistable, i.e., the apparatus is stable in two unconstrained shapes. The application of the invention illustrated in FIGS. 3A-C is only an example of apparatus that exhibits bistable behavior. It is noted that other bistable apparatus may be used in combination with this application of the invention.

Reference is now made to FIG. 3B. Once the adhesive 42 has bound to skin 20, the user applies a deforming force to apparatus 38. Typically, the application of this force comprises pushing down on one or more specific parts of apparatus 38, such as peripheral areas of mount 40, as illustrated by arrows 45. The deforming force causes mount 40 to move to a second configuration, e.g., a second unconstrained shape. Typically, this second unconstrained shape is concave toward adhesive 42, such that adhesive 42 is disposed within the concavity, and the skin is raised (as shown in FIG. 3C). Alternatively or additionally, the second unconstrained shape may be any shape that disposes adhesive 42 more superficially than does the first unconstrained shape. For example, the second unconstrained shape may be generally flat. The deforming force required to deform mount 40 in this manner is typically greater than 20 mN and may be greater than 100 mN. Typically, the deforming force required to deform mount 40 is not greater than 2000 mN. Typically, the distance the adhesive moves, from when the mount is in its unconstrained shape to when the adhesive contacts the skin, is greater than 3 mm and may be greater than 5 mm. Alternatively or additionally, the user may make the wrinkle shallower prior to deformation of apparatus 38 (e.g., by pulling the skin in a direction substantially orthogonal to that of the wrinkle), thereby reducing the distance that adhesive 42 moves before contacting the skin.

Reference is now made to FIG. 3C. Due to its adhesion to adhesive 42 of apparatus 38, the skin is automatically lifted superficially, as illustrated by arrows 46. In some applications of the invention, mount 40 is configured such that movement of mount 40 from the first to the second unconstrained shape is sudden. In some applications of the invention, mount 40 is configured such that this sudden movement is accompanied by a noise, e.g., a click. The click may be useful for indicating to the user that the operation of apparatus 38 has successfully caused the desired lifting of the skin.

Typically, the apparatus illustrated in FIGS. 2A-C and 3A-C is left on the skin for a sustained period (e.g., at least three hours, and/or less than 18 hours, e.g., overnight). During this period, the underlying skin is restructured by apparatus 38 in a manner that chronically treats the wrinkle. It is hypothesized that this sustained lifting of the skin has one or more of the following effects: induction of the influx of interstitial fluid and nutrients into the area of the lifted skin, improvement of skin hydration, stimulation of skin cells, and modulation of collagen in the treated area. Typically, treatments using apparatus as described herein are applied repeatedly (e.g., every day, every second day, or once per week) and/or over an extended period (e.g., for at least a week, a month, or a year). As described with reference to FIGS. 1A-B, for some applications, a kit is provided comprising more than one unit of apparatus 38, to facilitate multiple treatments of a wrinkle. For example, the kit may contain at least 5 units, and/or less than 40 units.

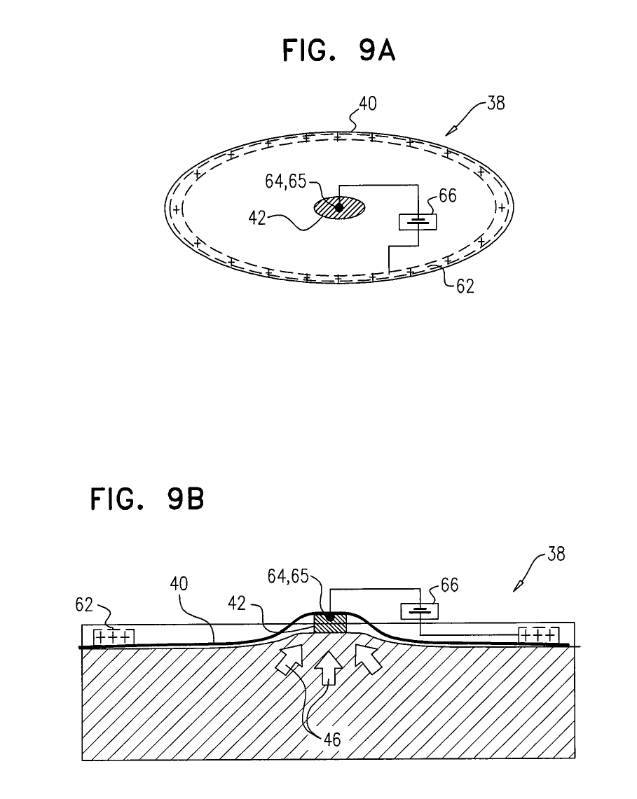

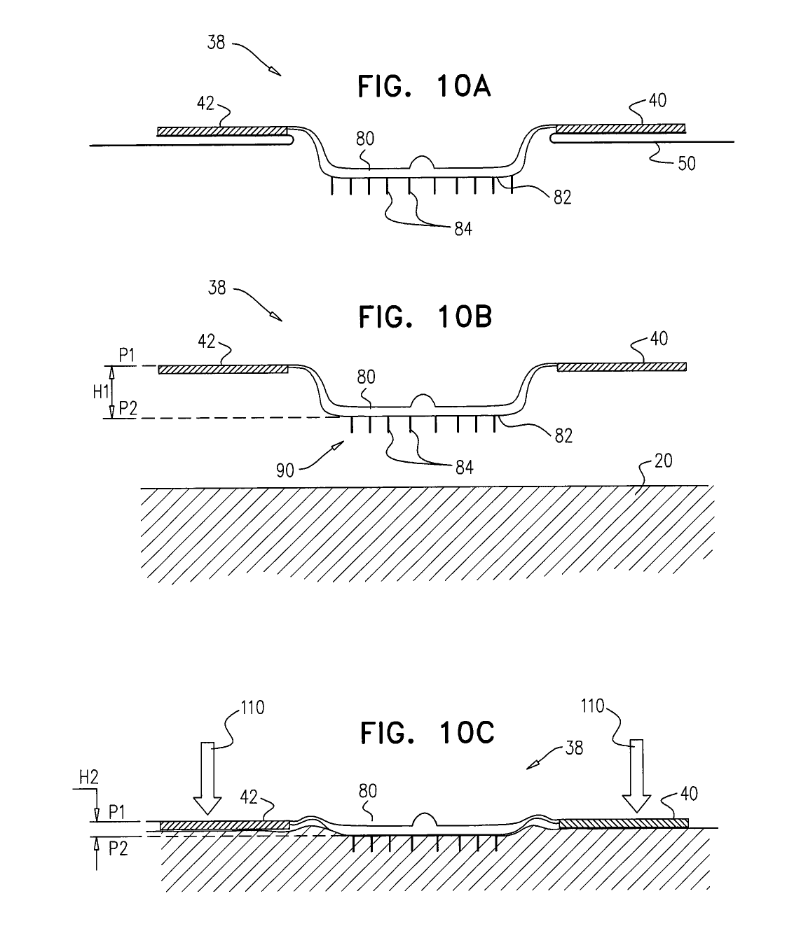

The applications of the invention illustrated in FIGS. 2A-C and 3A-C may be combined with the application of drugs and/or energy to increase the rate of skin restructuring. Examples include the application of a topical cream prior to application of apparatus 38, iontophoretic administration of a drug by apparatus 38 (as described with reference to FIGS. 9A-B), and the administration of healing light (e.g., red light), RF energy or ultrasound to the skin while apparatus 38 is on the skin.

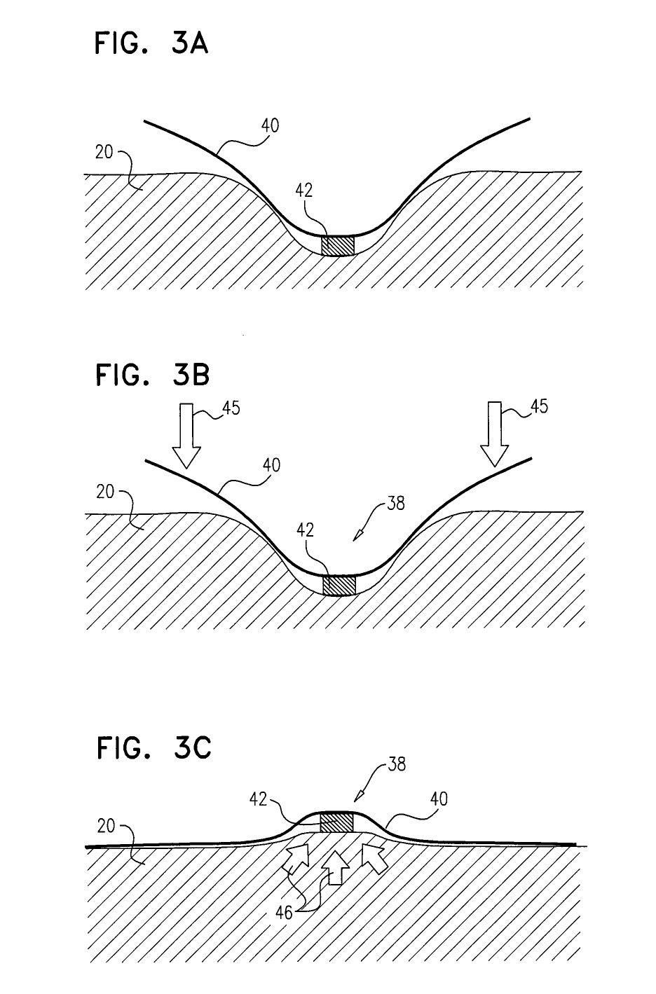

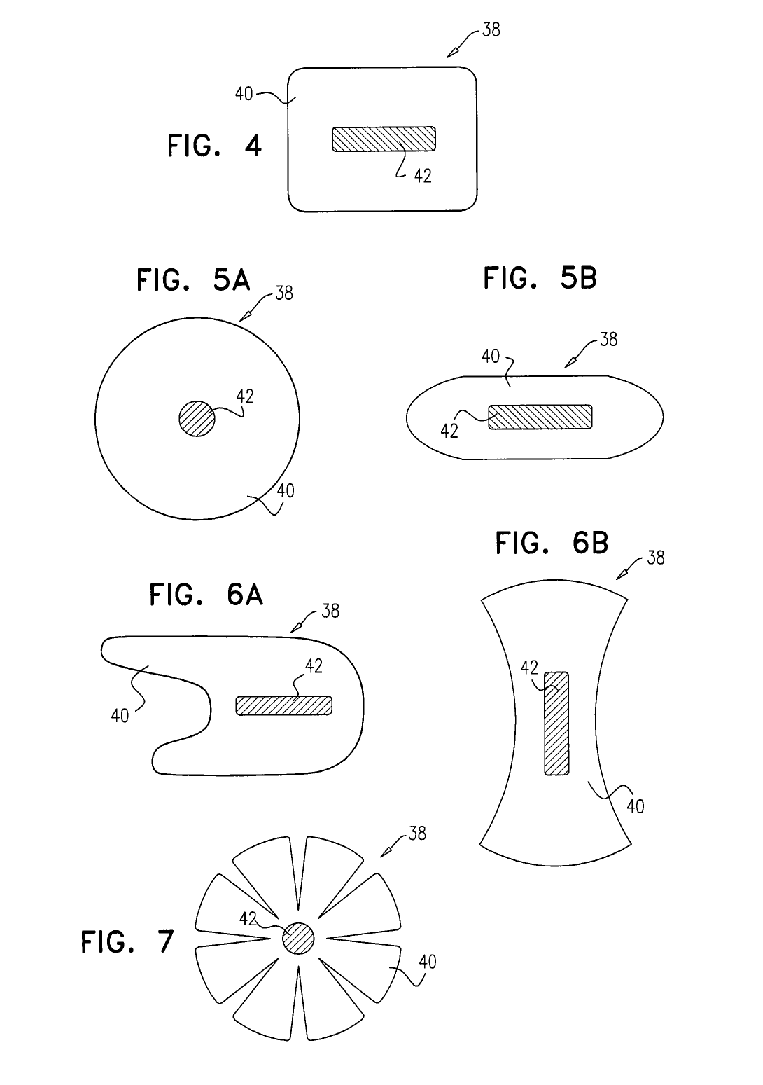

The applications of the invention described with reference to FIGS. 1A-B, 2A-C, and 3A-C may be used in combination with other applications of the invention described herein, including those described with reference to FIGS. 4, 5A-B, 6A-B, 7, 8A-D, 9A-B, and 12A-F.

Reference is now made to FIGS. 4, 5A-B, 6A-B and 7, which are schematic illustrations of apparatus 38, in accordance with several applications of the invention. These figures show the skin-facing side of mount 40 and adhesive 42. These figures are not intended to limit the scope of the invention but, rather, to illustrate variations of shapes and configurations of the invention as a whole, and of mount 40 and adhesive 42 individually. Suitably-shaped mounts may be selected, based on the skin area to be treated. For example, the mount shown in FIG. 6A or FIG. 6B may be placed laterally of a subject's eye, to treat “crow's-feet” wrinkles. The applications of the invention described with reference to FIGS. 4, 5A-B, 6A-B and 7 may be used in combination with other applications of the invention described herein, including those described with reference to FIGS. 1A-B, 2A-C, 3A-C, 8A-D, 9A-B, 10A-H and 12A-F.

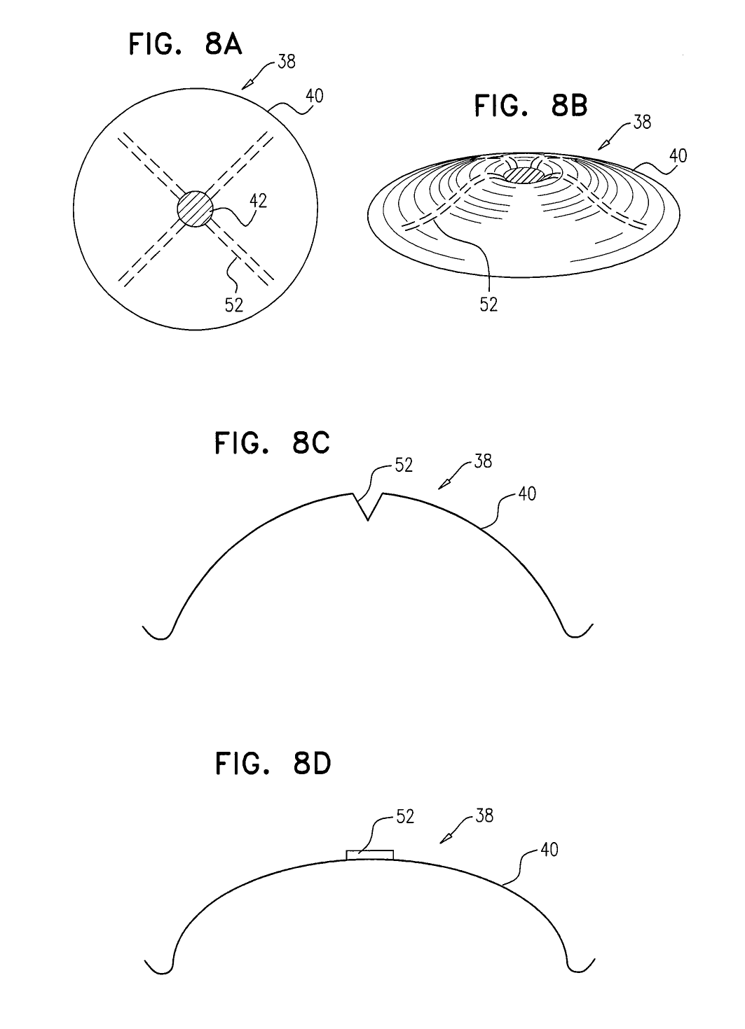

Reference is made to FIGS. 8A-D, which are schematic illustrations of apparatus 38, in accordance with an application of the invention. FIG. 8A provides a view of the skin-facing side of mount 40 and FIG. 8B provides a three-dimensional side-view representation of the outward-facing side. In this application of the invention, mount 40 comprises one or more structural ridges 52. Structural ridges increase the rigidity of mount 40 and thus increase the energy required to deform it. Alternatively or additionally, structural ridges 52 may increase the suddenness with which mount 40 changes shape. Structural ridges 52 may generate a sound (e.g., a click), or increase the sound generated, when mount 40 is deformed.

Reference is now made to FIGS. 8C-D. FIG. 8C is a cross-sectional representation of an application of the invention, where a structural ridge 52 is integrally formed in the material of mount 40 e.g., a structural ridge 52 is formed by incorporating a fold-like structure in the material of mount 40. As described hereinabove, mount 40 typically has a skin-facing side and an outward-facing side. In this application of the invention, mount 40 is shaped to define a curved edge on the skin-facing side. This curved edge is hypothesized to provide a smooth skin-contacting surface to prevent apparatus from leaving marks (e.g., imprints) in or on the skin. FIG. 8D is a cross-sectional representation of an application of the invention, where a structural ridge 52 is coupled to the material of mount 40 e.g., a structural ridge 52 is formed by adhering a strip to mount 40. In this application of the invention, mount 40 is shaped to define a curved edge on the skin-facing side. This curved edge is hypothesized to provide a smooth skin-contacting surface to prevent apparatus 38 from leaving marks (e.g., imprints) in or on the skin. In the applications of the invention described with reference to FIGS. 8C-D, In some applications of the invention, the apparatus described with reference to FIGS. 8C-D may not comprise structural ridges 52.

The applications of the invention described with reference to FIGS. 8A-D may be used in combination with other applications of the invention described herein, including those described with reference to FIGS. 1A-B, 2A-C, 3A-C, 4, 5A-B, 6A-B, 7, 9A-B, 10A-H and 12A-F.

Reference is now made to FIGS. 9A-B, which are schematic illustrations of apparatus 38, in accordance with an application of the invention. Apparatus 38 comprises mount 40 and a treatment unit, the treatment unit comprising adhesive 42. In this application of the invention, the treatment unit further comprises circuitry for use in iontophoretic administration of a drug, in accordance with another application of the invention. FIG. 9A provides a view of the skin-facing side of this application of the invention, and FIG. 9B provides a side view. In this application of the invention, the lifting mechanism of wrinkle treatment described hereinabove is accompanied by iontophoretic administration of a drug. A reservoir 64 contains a negatively-charged drug molecule to be administered; typically a water-soluble drug in liquid, gel (e.g., hydrogel) form. Reservoir 64 may be a hollow structure or an absorbent material and may be located in the same region of mount 40 as adhesive 42, or in another location. Alternatively or additionally, adhesive 42 may function as, and/or comprise, and/or replace reservoir 64, in which case, the adhesive itself includes the drug. Reservoir 64 typically comprises, or is coupled to, a cathode 65, which is connected to a power supply 66. The position of power supply 66 illustrated in FIG. 9B is for clarity only. Typically, power supply 66 is mounted on the apparatus. An anode 62 is connected to power supply 66, at another location on the skin-facing side of mount 40. The resulting electrical current repels the negatively-charged molecule from reservoir 64 into the skin. By reversing the polarity of the circuitry, positively-charged molecules may be administered in the same way, mutatis mutandis.

FIGS. 9A-B illustrate an application of the invention comprising a single reservoir 64, a single cathode 65, and a single anode 62. Other applications of the invention may comprise more than one of any of these elements. The applications of the invention described with reference to FIGS. 9A-B may be used in combination with other applications of the invention described herein, including those described with reference to FIGS. 1A-B, 2A-C, 3A-C, 4, 5A-B, 6A-B, 7, 9A-B, 10A-H and 12A-F.

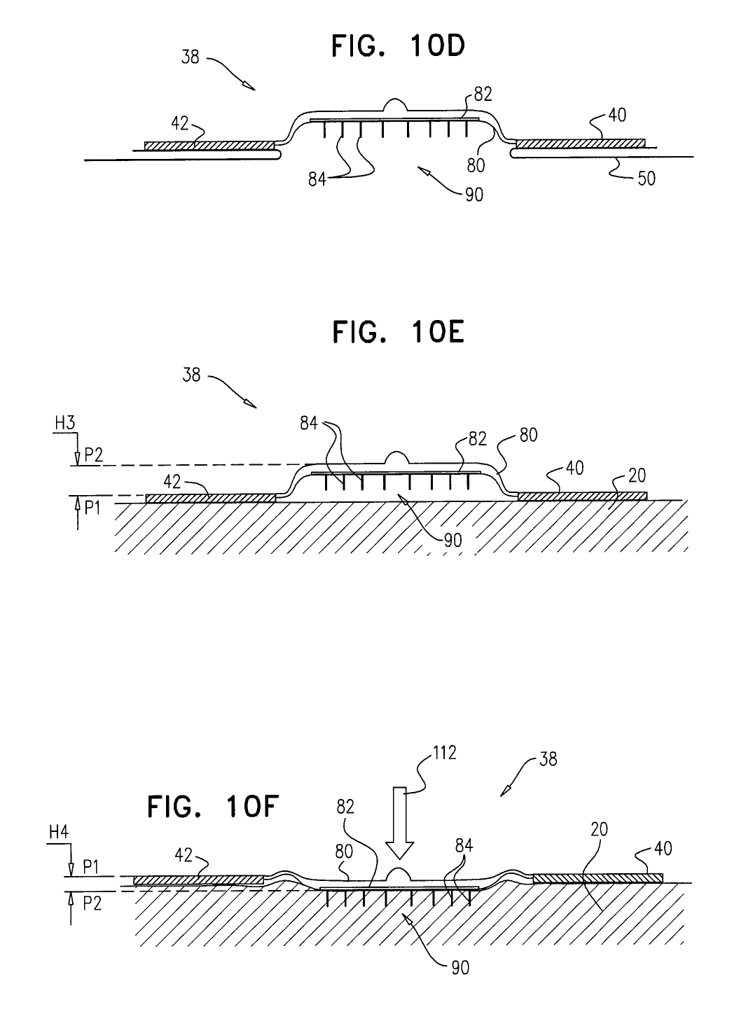

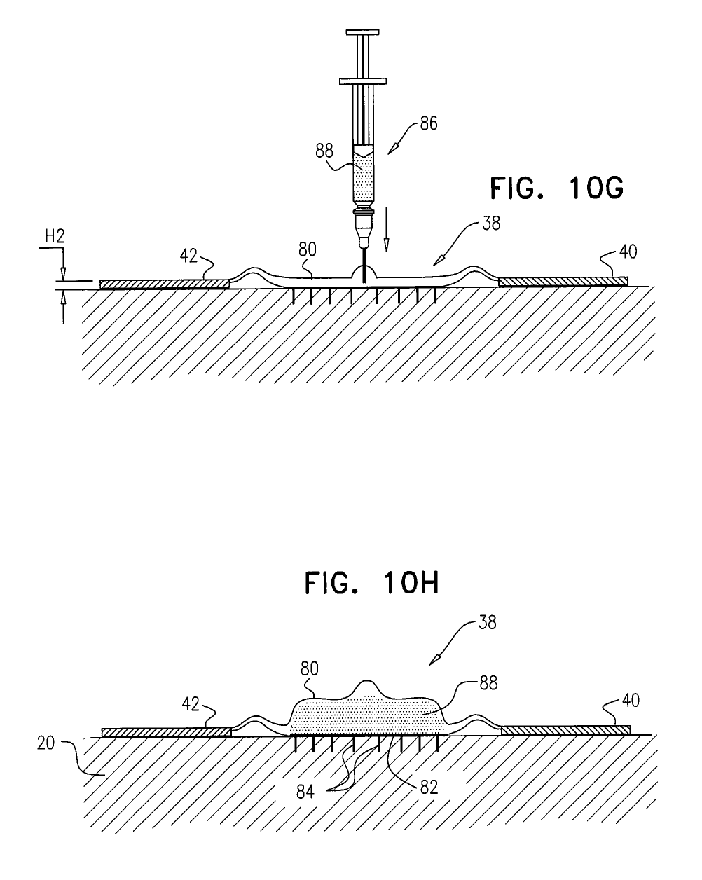

Reference is made to FIGS. 10A-H, which are cross-sectional schematic illustrations of apparatus 38, in accordance with respective applications of the invention. In accordance with these applications of the invention, apparatus 38 comprises mount 40 and a treatment unit, the treatment unit comprising a needle element, such as a microneedle unit 90. The microneedle unit typically comprises a reservoir 80, a plate 82 and a plurality of hollow microneedles 84 that extend from the apparatus. Alternatively, plate 82 may be an integral component of reservoir 80 (e.g., a wall of the reservoir), rather than a distinct element. Proximal ends of microneedles 84 are in fluid communication with reservoir 80, such that a substance can flow between the reservoir and distal ends of microneedles 84, into tissue of the subject (or, in the case of analyte extraction, out of the tissue of the subject).

Adhesive 42 is disposed on the skin-facing side of mount 40, in a location different from that of microneedle unit 90. Typically, but not necessarily, microneedle unit 90 is disposed at or near the center of mount 40, and adhesive 42 is disposed in one or more peripheral regions of the mount (e.g., at the edge of the skin-facing side of the mount). Adhesive 42 is protected by liner 50, which is removed prior to adhesion to the skin. Liner 50 may also protect microneedle unit 90. Alternatively, a separate component may protect the microneedle unit.

For purposes of illustration, mount 40 can be considered to reside substantially on two parallel planes, such that the areas of mount 40 on which adhesive 42 is disposed reside on a first plane P1, and the area of mount 40 on which microneedle unit 90 is disposed resides on a second plane P2. As described hereinabove, mount 40 has a skin-facing side and an outward-facing side. For clarity, apparatus 38 is described hereinbelow in the context of its orientation when applied to the skin, even with reference to figures that illustrate the apparatus prior to its application to the skin. The monostable/bistable paradigm described with reference to FIGS. 2A-C and 3A-C may also be applied to this application of the invention.

Reference is now made to FIGS. 10A-C, which are schematic illustrations of sequential steps in the use of apparatus 38 to administer a drug, in accordance with an application of the invention. FIG. 10A illustrates the application of the invention as supplied. FIG. 10B illustrates the application of the invention prior to its placement on the skin. In this application of the invention, mount 40 has a monostable configuration, with an unconstrained shape in which P1 is further from the skin than is P2. The height H1 between P1 and P2 in the unconstrained shape is typically less than 10 mm, and/or greater than 2 mm.

Reference is now made to FIG. 10C. Adhesive 42 is adhered to skin 20 by application of a force 110. Force 110 deforms mount 40 into a constrained shape in which P1 and P2 are brought closer together, such that the height H2 between P1 and P2 is smaller than H1. For example, H2 may be between 0 and 10 mm, e.g., less than 5 mm. Mount 40 is biased toward the unconstrained shape and, therefore, imparts a skinward force to microneedle unit 90, pressing and holding the microneedles 84 in the skin. Typically, therefore, the parts of microneedle unit 90 that contact the skin are disposed deeper than is adhesive 42. This improved contact is hypothesized to reduce the leakage that is associated with many microneedle patches, and/or to improve the stability of the microneedles in the skin.

Reference is now made to FIGS. 10D-F, which are schematic illustrations of sequential steps in the use of apparatus 38 to administer a drug, in accordance with an application of the invention. In this application of the invention, mount 40 has a bistable configuration, i.e., it has two unconstrained shapes. FIG. 10D illustrates the application of the invention as supplied.

Reference is now made to FIG. 10E. Apparatus 38 is supplied with mount 40 in a first unconstrained shape, in which P1 is closer to the skin than is P2. The height H3 between P1 and P2 is typically less than 10 mm and/or greater than 2 mm. Apparatus 38 is applied to skin 20, such that adhesive 42 adheres to the skin.

Reference is now made to FIG. 10F. A user applies a deforming force 112 to mount 42. For example, the user pushes down on a central region of the mount. The deforming force causes mount 40 to deform toward a second unconstrained shape, in which P2 is closer to the skin than in the first unconstrained shape. Typically, P2 is coplanar with P1 or is deeper than P1, such that microneedles 84 are pressed and held in the skin. Typically, therefore, the parts of microneedle unit 90 that contact the skin are disposed deeper than is adhesive 42. This improved contact is hypothesized to reduce the leakage that is associated with many microneedle patches and/or to improve the stability of the microneedles in the skin. This bistable configuration typically allows the apparatus 38 to be easily adhered to the skin, with deployment of microneedles 84 into the skin only once the apparatus is securely in place.

The deforming force required to deform mount 40 in this manner is typically greater than 20 mN and may be greater than 100 mN. Typically, the deforming force required to deform mount 40 is not greater than 2000 mN. Typically, the distance the adhesive moves, from when the mount is in its unconstrained shape to when the adhesive contacts the skin, is greater than 3 mm and may be greater than 5 mm. Alternatively or additionally, the user may make the wrinkle shallower prior to deformation of apparatus 38 (e.g., by pulling the skin in a direction substantially orthogonal to that of the wrinkle), thereby reducing the distance that adhesive 42 moves before contacting the skin.

In some applications of the invention, mount 40 is configured such that movement of mount 40 from the first to the second unconstrained shape is sudden. In some applications of the invention, mount 40 is configured such that this sudden movement is accompanied by a noise, e.g., a click. The click may be useful for indicating to the user that the operation of apparatus 38 has successfully pressed microneedles 84 into the skin.

Reference is now made to FIGS. 10G-H. Following adhesion of apparatus 38 to skin 20, drug 88 is administered to reservoir 80. Typically, a syringe 86 is used for this purpose. Reservoir 80 typically comprises an elastic material, such that the reservoir is expandable upon filling and such that the return of the reservoir toward its original configuration applies pressure to drug 88, which is then forced through microneedles 84 into skin 20. Alternatively, reservoir 80 may have a fixed configuration.

In an application of the invention, reservoir 80 is supplied pre-filled, or may be filled by the user prior to adhesion to the skin.

The configurations of mount 40 and adhesive 42 described with reference to FIGS. 10A-F, and which typically cause a skin-contact part of a treatment unit to be pressed into the skin deeper than is adhesive 42, may also be employed in combination with other treatment elements where improved skin contact is beneficial. For example, apparatus 38 may comprise a mount which presses anode 62 and cathode 65 into the skin, thereby increasing electrical contact with the skin, in combination with the iontophoresis circuitry described with reference to FIGS. 9A-B. Similarly, apparatus 38 may be used to press drugs in forms such as liquids, creams, gels or powders, onto the skin, typically to provide greater contact than that provided by existing skin patches.

The applications of the invention described with reference to FIGS. 10A-H are used, for some applications, in combination with other needle-based devices. For example, apparatus 38 may be used to administer a drug via a single needle, or via a plurality of needles which are not microneedles. Alternatively, microneedle unit 90 may comprise solid (i.e., not hollow) needles, that do not facilitate drug delivery. For example, apparatus 38 may press solid needles into the skin, such as for the stimulation of tissue of the subject, a method commonly referred to as “needling.”

In a further application of the invention, microneedle unit 90 may be used for collecting an analyte, rather than for administering a drug. For example, reservoir 80 may comprise a vacuum and/or an absorbent material. The applications of the invention described with reference to FIGS. 10A-H may be used in combination with other applications of the invention described herein, including those described with reference to FIGS. 4, 5A-B, 6A-B, 7, 8A-D, 9A-B, and 11A-H.

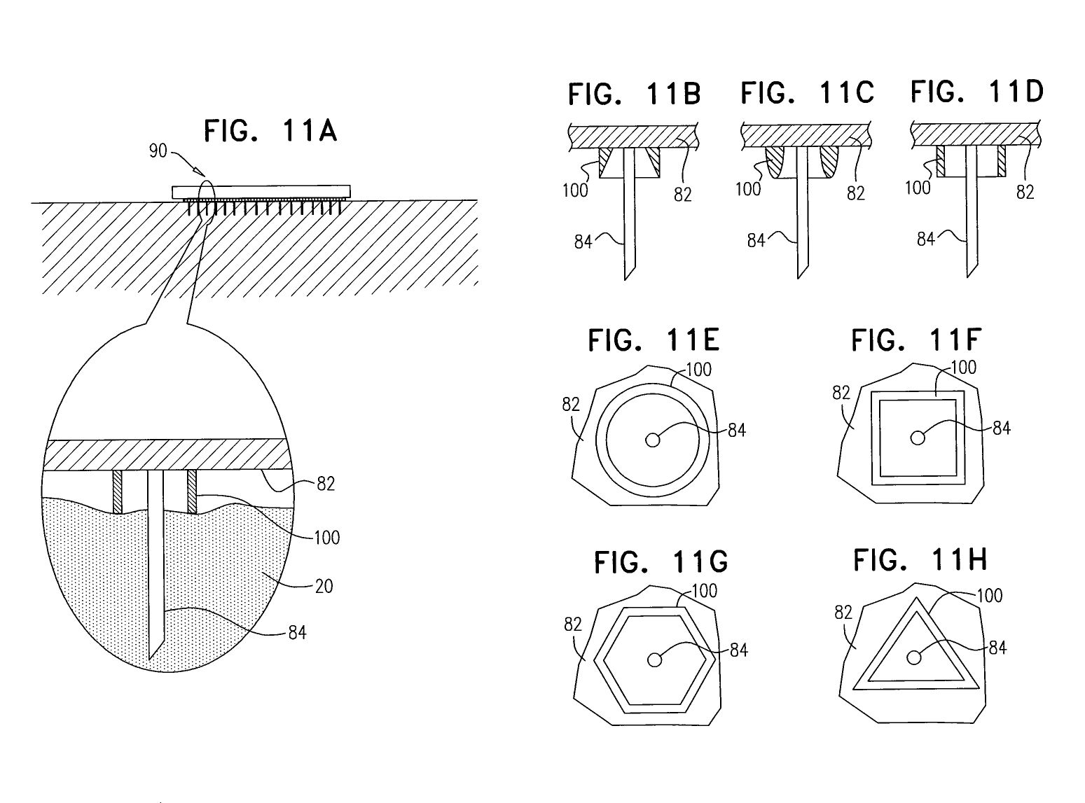

Reference is made to FIG. 11A. A seal 100 is situated around the base of each microneedle 84. Seal 100 reduces leakage onto the surface of skin 20 of a drug 88 that is released from microneedle 84. The total surface area with which the seals contact the skin is relatively small compared to that of a flat microneedle plate with no such seals and, therefore, the pressure at the point of contact with skin 20 is higher when seals 100 are employed than in the absence of the seals.

Reference is now made to FIGS. 11B-H, which illustrate applications of seal 100, in accordance with several applications of the invention. Ridge 100 has a toroid or toroidal polyhedron shape i.e., it is generally similar to a ring or short tube but may have one of many face shapes and cross-sectional shapes. FIGS. 11B-D are schematic illustrations of several applications of the invention, illustrating several of the cross-sectional shapes that seal 100 may have: triangular (FIG. 11B), rounded (FIG. 11C) and rectangular (FIG. 11D). FIGS. 11E-H are schematic illustrations of several applications of the invention, showing several of the face shapes that seal 100 may have: circular (FIG. 11E), square (FIG. 11F), hexagonal (FIG. 11G) and triangular (FIG. 11H). The shapes illustrated in FIGS. 11B-H are by no means intended to limit the scope of the invention but, rather, to illustrate the possible variation of shapes and configurations of the invention. The choice of shape for seal 100 will likely be dependent on the specific demands of a given application. For example, seals with a hexagonal face shape, such as illustrated in FIG. 11G, will tessellate well, allowing a high density of microneedles 84 on plate 82.

The application of the invention described with reference to FIGS. 11A-H may also be used for the application of any needle-based device. For example, a skin-attached device that administers a drug via a hypodermic needle. The applications of the invention described with reference to FIGS. 11A-H may be used in combination with other applications of the invention described herein, including those described with reference to FIGS. 4, 5A-B, 6A-B, 7, 8A-D, 9A-B, and 10A-H.

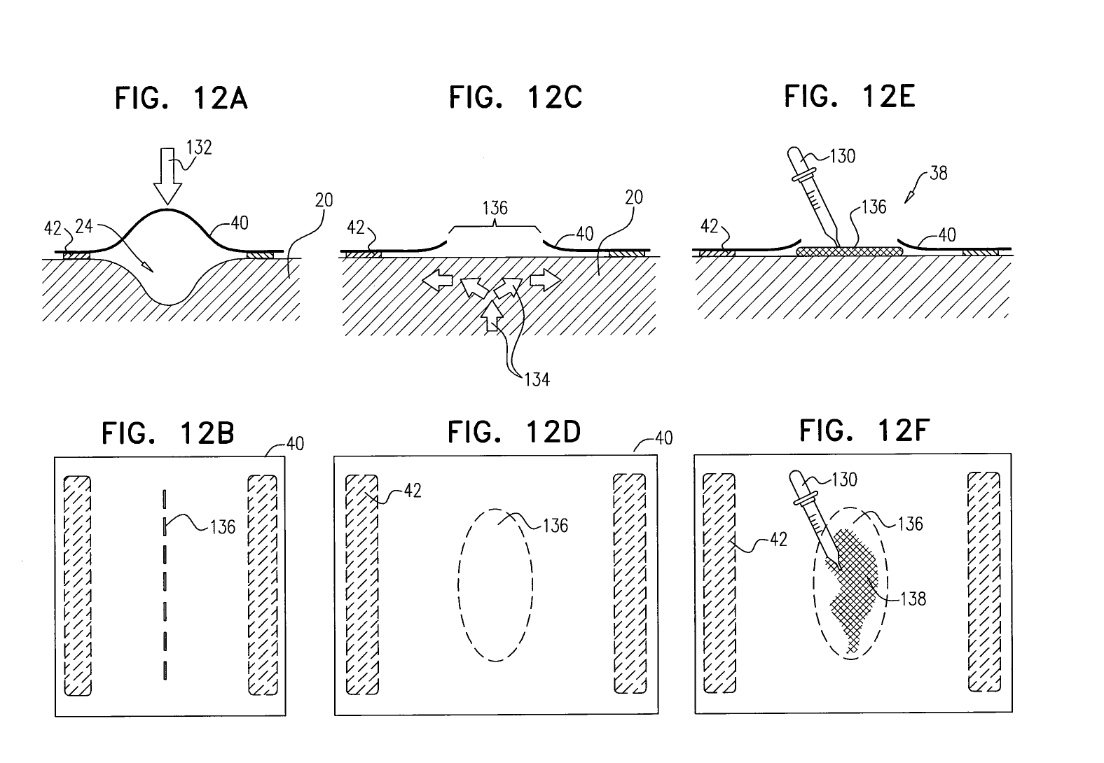

Reference is made to FIGS. 12A-F, which are schematic illustrations of the use of apparatus 38 to treat a wrinkle, in accordance with an application of the invention. FIGS. 12A, 12C, and 12E are cross-sectional views of sequential steps in the treatment; FIGS. 12B, 12D, and 12F illustrate respective top views of the apparatus.

Reference is made to FIGS. 12A-B. One or more adhesives 42 are disposed on the skin-facing side of mount 40. Mount 40 is adhered to skin 20 of the subject while the mount is in a first shape. Typically, two adhesives 42 are disposed on the skin-facing side of the mount and are positioned on either side of wrinkle 24. Mount 40 is shaped to provide an opening 136. In some applications of the invention, when the mount is in the first shape, opening 136 is substantially closed (e.g., it is a slit or it is a weakened zone e.g., a perforated zone or a zone of thinner material). In other applications of the invention, opening 136 is substantially open. A deforming force is applied to mount 40. For example, a user may push down on a central region of the mount, as indicated by arrow 132.

Reference is now made to FIGS. 12C-D. The deforming force causes mount 40 to assume a second shape, such that adhesives 42 are further apart, thereby stretching and reducing the wrinkle, as illustrated by arrows 134. The mount is typically configured such that the mount remains in the second shape after removal of the deforming force. In the example hereinabove, the two adhesives positioned on either side of the wrinkle are moved apart, thereby stretching the wrinkle in a direction that is generally orthogonal to the direction of the wrinkle and making the wrinkle shallower. Deformation of the mount from the first shape to the second shape may also increase the size of opening 136. Alternatively, opening 136 may remain substantially the same size between the two shapes of mount 40.

In an alternative application of the invention, mount 40 may not change shape; rather the wrinkle is made shallower by the user (e.g., by pulling the skin in a direction substantially orthogonal to that of the wrinkle) and mount 40 is then adhered to the skin to hold the wrinkle in the shallower configuration.

Reference is now made to FIGS. 12E-F. The wrinkle remains held in the shallower position by mount 40 and is accessible through opening 136. A substance that can be adhered to the skin and become generally resilient, e.g., glue 138 is applied to the wrinkle through opening 136 using an applicator 130. Glue 138 may comprise cyanoacrylate, latex, or any other suitable ingredient. Glue 138 may be biological (e.g., collagen-based). Glue 138 is typically applied in a fluid form (e.g., as a liquid). Alternatively, glue 138 may be applied as a gel, a paste, an aerosol, or a flexible patch. Applicator 130 may comprise a syringe, a dropper, a sprayer, a brush, a spreader, a pen, or any other suitable device for applying glue 138 to the skin. Following application of glue 138 to the wrinkle, the glue becomes firmer and/or stiffer over a period of minutes (e.g., 60 min, 30 min, 15 min, 5 min, or 1 min). Glue 138 adheres to skin 20 such that it holds the wrinkle in its shallow configuration. Mount 40 is typically subsequently removed, leaving only glue 138 on the skin.

Typically, glue 138 is left on the skin for a sustained period (e.g., at least 30 minutes, and/or less than 18 hours, e.g., overnight). During this period, the underlying skin is restructured to support the lifted configuration. Typically, treatments using apparatus as described herein are applied repeatedly (e.g., every day, every second day, or once per week) and/or over an extended period (e.g., for at least a week, a month, or a year). For some applications, a kit is provided comprising more than one unit of apparatus 38, to facilitate multiple treatments of a wrinkle. For example, the kit may contain at least 5 units, and/or less than 40 units.

In applications of the invention where glue 138 is biological and/or biodegradable, the glue may be left on the skin for a longer sustained period (e.g., more than a day, e.g., more than a week). In these applications of the invention, glue 138 may not be actively removed; rather it may be absorbed by the skin or degraded on the skin. It is hypothesized that absorption of collagen-based biological glue 138 by the skin enhances treatment of the wrinkle.

The application of the invention illustrated in FIGS. 12A-F may be combined with the application of drugs and/or energy waves to increase the rate of skin restructuring. Examples include the application of a collagen-expression-inducing topical cream to the wrinkle prior to application of glue 138, and the application of radio or sound waves (e.g., ultrasound) to the skin while the apparatus is on the skin. The applications of the invention described with reference to FIGS. 12A-F may be used in combination with other applications of the invention described herein, including those described with reference to FIGS. 4, 5A-B, 6A-B, 7, 8A-D, 9A-B, and 10A-H.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for use with skin of a subject, comprising:
a mount, a first zone of which is adhesively adherable to the skin and a second zone of which is not adhesively adherable to the skin, the mount being disposable in one shape and in another shape, at least one of the shapes being an unconstrained shape, the mount being deformable from the one shape to the other shape in response to application of a deforming force to the mount, and configured such that when the mount moves toward the unconstrained shape, the movement is sudden regardless of a speed of application of the deforming force prior to the sudden movement; and
a treatment unit coupled to the mount, positioned with respect to the mount such that the treatment unit is moved into a position to apply a treatment to the subject by the mount moving toward the unconstrained shape.

2. The apparatus according to claim 1, wherein the skin of the subject to which the first zone of the mount is adhesively adherable has a plane, and wherein the treatment unit is positioned with respect to the mount such that the mount moving toward the unconstrained shape, moves the treatment unit in a direction that is substantially orthogonal to the plane.

3. The apparatus according to claim 1, wherein the one shape is the unconstrained shape, and wherein the mount has sufficient resilience to return toward the one shape after removal of the deforming force.

4. The apparatus according to claim 3, wherein the first zone of the mount is configured to be disposed more superficially when the mount is adhered to the skin and in the unconstrained shape than when the mount is adhered to the skin and in the other shape.

5. The apparatus according to claim 1, wherein the other shape is the unconstrained shape and the one shape is an unconstrained shape, and wherein the mount is configured such that it remains in the other shape after removal of the deforming force.

6. (canceled)

7. The apparatus according to claim 5, wherein the first zone of the mount is configured to be disposed more superficially when the mount is adhered to the skin and is in the other shape than when the mount is adhered to the skin and is in the one shape.

8. The apparatus according to claim 1, wherein:
the treatment unit comprises an adhesive, and
the adhesive is configured to apply the treatment by being positioned with respect to the mount such that when the mount is adhered to the skin, the mount moving toward the unconstrained shape moves the adhesive superficially.

9.-19. (canceled)

20. The apparatus according to claim 1, wherein the first zone of the mount comprises at least part of a skin-facing side of the mount, configured to face skin of the subject, and wherein the second zone of the mount comprises an outward-facing side of the mount, configured to face away from skin of the subject.

21. The apparatus according to claim 20, wherein the first zone of the mount is disposed in a central region of the skin-facing side of the mount.

22. The apparatus according to claim 21, wherein the first zone of the mount is not disposed at an edge of the skin-facing side of the mount.

23. The apparatus according to claim 20, wherein the first zone of the mount is disposed in a peripheral region of the skin-facing side of the mount.

24. The apparatus according to claim 20, wherein the first zone of the mount is not disposed in a central region of the skin-facing side of the mount.

25. The apparatus according to claim 1, wherein the mount is configured such that it is deformable from the one shape to the other shape only when the deforming force is at least 20 mN.

26. The apparatus according to claim 25, wherein the mount is configured such that it is deformable from the one shape to the other shape only when the deforming force is at least 100 mN.

27. The apparatus according to claim 1, wherein the mount is configured such that when at least one element of the mount is stationary and the mount moves to the unconstrained shape, the treatment unit moves at least 3 mm.

28. The apparatus according to claim 27, wherein the mount is configured such that when the element of the mount is stationary and the mount moves to the unconstrained shape, the treatment unit moves at least 5 mm.

29. The apparatus according to claim 27, wherein the mount is configured such that when the element of the mount is stationary and the mount moves to the unconstrained shape, no part of the mount moves greater than 40 mm.

30. The apparatus according to claim 29, wherein the mount is configured such that when the element of the mount is stationary and the mount moves to the unconstrained shape, no part of the mount moves greater than 25 mm.

31. The apparatus according to claim 29, wherein the mount is configured such that it is deformable from the one shape to the other shape only when the deforming force is at least 20 mN, and wherein the mount is configured such that it is deformable from the one shape to the other shape even when the deforming force is less than 2000 mN.

32.-42. (canceled)

43. A method for use with a wrinkle of skin of a subject, the method comprising:
adhering apparatus to the wrinkle of the subject, the apparatus including a mount and an adhesive;
deforming the mount from one shape toward another shape by applying a deforming force to the mount, at least one of the shapes being an unconstrained shape; and
inducing a sudden movement of the mount toward the unconstrained shape, such that the adhesive moves superficially with respect to the skin, the suddenness of the movement being regardless of a speed of application of the deforming force prior to the sudden movement.

44. The method according to claim 43, wherein deforming the mount from the one shape toward the other shape comprises deforming the mount from an unconstrained shape toward a constrained shape, and wherein inducing sudden movement of the mount toward the unconstrained shape comprises removing the deforming force, the mount having sufficient resilience to return toward the unconstrained shape after removal of the deforming force.

45. The method according to claim 43, wherein deforming the mount from the one shape toward the other shape comprises deforming the mount from one unconstrained shape toward another unconstrained shape.

46.-51. (canceled)

52. A method for use with skin of a subject, the method comprising:
adhering apparatus to the skin of the subject, the apparatus including a mount and a treatment unit coupled to the mount; and
deforming the mount from one shape to another shape by applying a deforming force to the mount, at least one of the shapes being an unconstrained shape; and
inducing a sudden movement of the mount toward the unconstrained shape, such that the treatment unit is moved into a position to apply a treatment to the subject.

53. The method according to claim 52, the skin of the subject to which the apparatus is adhered having a plane, and wherein inducing the sudden movement of the mount toward the unconstrained shape such that the treatment unit is moved into a position to apply a treatment to the subject, comprises moving the treatment unit in a direction that is substantially orthogonal to the plane.

54. The method according to claim 52, wherein deforming the mount from the one shape into the other shape comprises deforming the mount from an unconstrained shape toward a constrained shape, and wherein inducing sudden movement of the mount toward the unconstrained shape comprises removing the deforming force, the mount having sufficient resilience to return toward the unconstrained shape after removal of the deforming force.

55. The method according to claim 54, wherein inducing the sudden movement of the mount such that the treatment unit is moved into the position to apply the treatment to the subject comprises inducing a sudden superficial movement of the treatment unit, the treatment unit including an adhesive.

56. The method according to claim 52, wherein deforming the mount from the one shape into the other shape comprises deforming the mount from one unconstrained shape into another unconstrained shape, the mount being configured such that it remains in the other unconstrained shape after removal of the deforming force.

57. The method according to claim 56, wherein inducing the sudden movement of the mount such that the treatment unit is moved into the position to apply the treatment to the subject comprises inducing a sudden movement of the mount such that the treatment unit is moved superficially, the treatment unit including an adhesive.

58.-82. (canceled)
